(19) United States
(12) Patent Application Publication
(10) Pub. No.: US 2020/0028172 A1
(43) Pub. Date: Jan. 23, 2020

(54) BIS(PYRIDINIUM)-NAPHTHALENE DIIMIDE REDOX IONIC COMPOUNDS AS ELECTRODE ACTIVE MATERIALS

(71) Applicants: BLUE SOLUTIONS, ERGUE GABERIC (FR); CENTRE NATIONAL DE LA RECHERCHE SCIENTIFIQUE, PARIS (FR); UNIVERSITE DE NANTES, NANTES (FR)

(72) Inventors: Marc DESCHAMPS, QUIMPER (FR); Joel GAUBICHER, NANTES (FR); Dominique GUYOMARD, SAUTRON (FR); Philippe POIZOT, TREILLIERES (FR); Margaud LECUYER, COMBRIT (FR); Sofia PERTICARARI, NANTES (FR); Fabrice ODOBEL, NANTES (FR)

(21) Appl. No.: 16/490,978

(22) PCT Filed: Mar. 7, 2018
(86) PCT No.: PCT/FR2018/050529

§ 371 Date: Sep. 4, 2019

(30) Foreign Application Priority Data
Mar. 8, 2017 (FR) 17 51907

(51) Int. Cl. H01M 4/60; H01M 10/0564; H01M 4/62; C07D 471/06

(52) U.S. Cl. CPC H01M 4/60; H01M 10/0564; H01M 2004/027; H01M 4/624; C07D 471/06; H01M 4/622

(57) ABSTRACT

The invention relates to the use of a bis(pyridinium)-naphthalene diimide redox ionic compound as electrode active material, notably for an aqueous electrolyte battery, to a negative electrode comprising at least said bis(pyridinium)-naphthalene diimide redox ionic compound, to a battery, notably an aqueous electrolyte battery comprising said negative electrode, and to particular bis(pyridinium)-naphthalene diimide redox ionic compounds.

The invention relates to the use of a bis(pyridinium)-naphthalene diimide redox ionic compound as electrode active material, notably for an aqueous electrolyte battery, to a negative electrode comprising at least said bis(pyridinium)-naphthalene diimide redox ionic compound, to a battery, notably an aqueous electrolyte battery comprising said negative electrode, and to particular bis(pyridinium)-naphthalene diimide redox ionic compounds.

The invention applies typically, but not exclusively, to the field of aqueous electrolyte batteries.

Batteries have become indispensable constituents in stationary and portable applications, such as portable electronic devices, electrical or mechanical appliances. They are also widely studied for use in electric vehicles and also in the field of energy storage. Currently, several technologies exist on the market such as the lead-acid system, dating from the beginning of the 20th century, which is still used in motor vehicle starter systems and also for numerous industrial or general use applications where the weight and size are not key criteria; the nickel-cadmium system, more widely deployed from the 1950s and used in “cordless” appliances, which is tending to disappear owing to the toxic nature of the cadmium but is still used for specific industrial applications such as aeronautics; the nickel-metal hydride system, marketed at the beginning of the 1990s and which was introduced into hybrid vehicles; and finally the lithium-ion (Li-ion) system, introduced in 1991, which now tends to be used for all applications notably owing to the high bulk energy densities obtained. The limitations of Li-ion systems are mainly linked to their cost which is still relatively high, stemming in part from the materials forming them, such as the inorganic electrode active materials and the organic (i.e. non-aqueous) electrolytes.

For historic reasons, but also reasons of electrochemical performance, the technologies currently marketed are based on the almost exclusive use of inorganic electrode active materials, mainly based on transition metals such as Co, Mn, Ni or Fe. However, these inorganic electrode active materials (e.g. LiCoO₂, LiMnO₄, LiFePO₄, LiNi_{0.8}Co_{0.15}Al_{0.05}O₂, LiNi_{1/3}Mn_{1/3}Co_{1/3}O₂, etc.) have many drawbacks such as the risk of explosion of the battery, their high toxicity, the problem of recycling them, their high cost and/or their low specific capacity. Moreover, these inorganic active materials are generally produced from resources of geological (i.e. non-renewable) origin and are energy consuming in their process. In view of the production volumes forecast for batteries (several billion units per year for Li-ion technology), there is a risk of these inorganic electrode active materials no longer being available in a large amount over time. Furthermore, none of the existing technologies fully meets the requirements, while new environmental standards are appearing at the European level (see http://ec.europa.eu/environment/waste/batteries/, directive 2006/66/EC).

In this context, the development of batteries comprising, as electrode active material, a redox organic structure (e.g. nitroxide derivatives, polyaromatic compounds), that is to say an organic structure capable of carrying out one or more reversible electrode reactions, allows certain possibilities to be anticipated. First of all, these redox organic structures have the advantage of comprising chemical elements (C, H, N, O, S, in particular) which can potentially derive from renewable resources, thus rendering them more plentiful. Next, they are destroyed fairly easily by simple combustion at relatively moderate temperature. In addition, their electrochemical properties (ion and electron conduction properties, redox potential, specific capacity) can be adjusted by appropriate functionalization (e.g. incorporation of attracting groups close to the redox centre for adjusting the potential).

In particular, Yao et al. [Int. J. of Electrochem. Sci., 2011, 6, 2905] described an organic lithium battery comprising a negative electrode consisting of a lithium metal foil; a positive electrode consisting of an aluminium current collector supporting an electrode material comprising 5,7,12,14-pentacenetetrone (PT) as active material, acetylene black as agent imparting electron conductivity and polytetrafluoroethylene as binder; a liquid electrolyte consisting of lithium bis(trifluoromethanesulfonyl)imide (LiTFSI) as a 1 mol/l solution in γ-butyrolactone; and a glass fibre separator impregnated with said liquid electrolyte. However, the cycling resistance of such a battery remains low since the initial specific capacity is of the order of 300 mAh/g and drops to 170 mAh/g after 10 cycles. This poor cycling stability is mainly linked to the solubility of the positive electrode active material (PT) in the solvent of the organic electrolyte (γ-butyrolactone). Indeed, most redox organic structures are soluble in the solvent of the organic electrolyte. Consequently, when a redox organic structure is used as electrode active material, the electron conductivity between the current collector and said active material becomes insufficient and the reactivity is reduced. In addition, the concentration of active material which may be involved in an electrode reaction is decreased, which causes a drop in the capacity of the battery. Finally, an organic electrolyte has an ion conductivity between ten and a hundred times lower than an aqueous electrolyte and it is not generally very environmentally friendly.

Thus, batteries combining an aqueous electrolyte (notably at pH 6-7) and at least one electrode based on a hybrid active material of metal-organic type have been proposed. In particular, US 2014/0220392 described a battery with an aqueous electrolyte (e.g. NaNO₃, KNO₃, NaClO₄, etc.) comprising a Prussian blue derivative (i.e. derivative of a ferric ferrocyanide of chemical formula Fe₇(CN)₁₈(H₂O)ₓ, in which x varies from 14 to 18) as positive and/or negative electrode active material. However, the specific capacity obtained is moderate (i.e. 55-60 mAh/g) and the aqueous electrolyte must be highly concentrated in salts in order to prevent the solubilization of the Prussian blue derivative in said aqueous electrolyte and/or the production of oxygen.

At the same time, an ionic compound based on viologen and on perylene diimide satisfying the following formula:

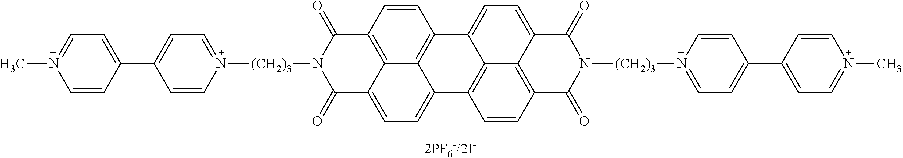

was used in an electrochromic cell for its properties of changing colour as a function of the potential [Kim et al., J. Mater. Chem., 2012, 22, 13558-13563J. However, this ionic compound is not used as an electrode active material, notably in an aqueous electrolyte battery.

Thus, the objective of the present invention is to overcome the drawbacks of the aforementioned prior art and to provide a battery in which the constituents are chosen so as to yield, at the lowest possible cost, a good electrochemical performance in terms of energy density and/or power performance, a good cycling stability and a certain safety.

In particular, there is a need for economical aqueous electrolyte batteries, which use inexpensive, recyclable and non-toxic raw materials, and which have good electrochemical performance, notably in terms of energy density and/or power performance and/or resistance to cycling.

These objectives are achieved by the invention which will be described hereinbelow.

A first subject of the invention is therefore the use of a redox ionic compound comprising at least one naphthalene diimide unit and at least one N,N′-disubstituted bis(pyridinium) unit, as negative electrode active material, notably in an aqueous electrolyte battery.

The naphthalene diimide unit may also be denoted by the term “naphthalene-1,4,5,8-tetracarboxylic diimide unit”.

The N,N′-disubstituted bis(pyridinium) unit is a unit that comprises two pyridinium moieties, each of the pyridinium moieties being substituted at the nitrogen atom. The two pyridinium moieties may be directly linked to form a bipyridine unit, and in particular a 4,4′-bipyridine unit, or may be linked by means of an appropriate group, in particular one or more alkenyl groups.

Thus, such a redox ionic compound is capable of reacting electrochemically in a reversible manner, and more particularly of carrying out one or more reversible redox reactions, notably by exchanging electrons with an electrode and simultaneously by combining with cations and/or anions.

The redox ionic compound used in the invention is preferably capable of exchanging anions, notably at a low voltage (i.e. less than or equal to 0 volts vs. SCE (saturated calomel electrode)).

The redox ionic compound used in the invention comprises two types of organic units: at least one p-type unit (N,N′-disubstituted bis(pyridinium)) and at least one n-type unit (naphthalene diimide). Owing to this combination of p- and n-type units, the compound has an advantageous electrochemical performance, notably in terms of cyclability and capacity, to be able to be used as electrode active material, notably in an aqueous electrolyte battery.

The N,N′-disubstituted bis(pyridinium) and naphthalene diimide units may be coupled within the redox ionic compound by means of a linker denoted by L. Thus, the linker L makes it possible to covalently bond the two types of units within the redox ionic compound.

The nature of the linker L is not critical. The linker L may be a saturated or unsaturated carbon chain, an aromatic carbon chain, or a mixture of a saturated or unsaturated carbon chain and an aromatic carbon chain, the aforementioned carbon chains being optionally fluorinated, and possibly containing one or more heteroatoms, for example one or more oxygen or sulfur atoms, said carbon chains having from 2 to 20 carbon atoms, preferentially from 2 to 10 carbon atoms, preferentially from 3 to 6 carbon atoms, and more preferentially still 2, 3, 4 or 5 carbon atoms.

The carbon chain is preferably saturated or unsaturated, and more preferably saturated.

According to one particularly preferred embodiment of the invention, the linker L is an alkylene chain, which is preferably linear, having from 2 to 10 carbon atoms, preferably from 3 to 6 carbon atoms, and more preferentially still 3, 4 or 5 carbon atoms.

An N,N′-disubstituted bis(pyridinium) unit may be represented by any one of the chemical formulae (I-a), (I-b) or (I-c) below:

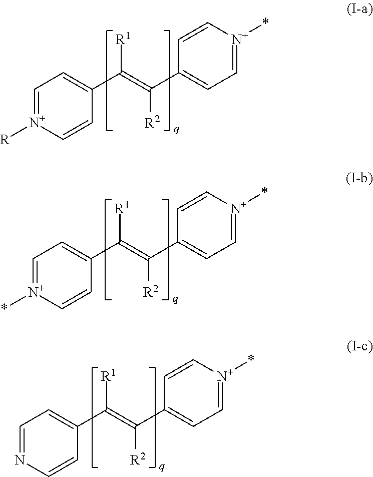

in which the sign * denotes the attachment point of the N,N′-disubstituted bis(pyridinium) unit to a naphthalene diimide unit, notably via the linker Las defined above; R represents an end group chosen from an alkyl group, an alkenyl group, an alkynyl group, and an aryl group, said aforementioned groups being optionally substituted by one or more aromatic groups, optionally fluorinated or perfluorinated, said aforementioned groups possibly containing one or more heteroatoms, for example one or more oxygen, sulfur or nitrogen atoms; R′ and R², which are identical or different, represent an alkyl group or a cyano group; and q is such that 0 q 4.

Indeed, the N,N′-disubstituted bis(pyridinium) unit may be bonded to two naphthalene diimide units as is the case when it satisfies the formula (I-b) or to a single naphthalene diimide unit as is the case when it satisfies one of the formulae (I-a) or (I-c).

Furthermore, when q is equal to zero, the 2 pyridinium moieties are bonded together directly, and when 1 q 4, the 2 pyridinium moieties are bonded together via one or more alkenyl groups.

A naphthalene diimide unit may be represented by either one of the chemical formulae (II-a) or (II-b) below:

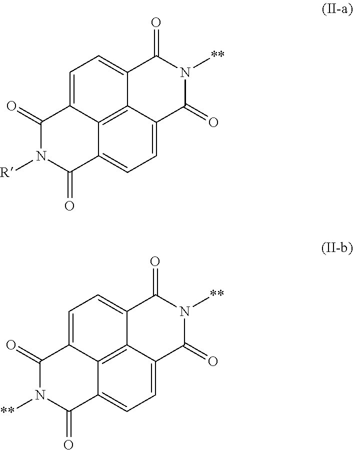

in which the sign ** denotes the attachment point of the naphthalene diimide unit to an N,N′-disubstituted bis(pyridinium) unit, notably via the linker L as defined above, and R′ represents a group chosen from a hydrogen atom, an alkyl group, an alkenyl group, an alkynyl group, and an aryl group, said aforementioned groups being optionally substituted by one or more aromatic groups, optionally fluorinated or perfluorinated, said aforementioned groups possibly containing one or more heteroatoms, for example one or more oxygen, sulfur or nitrogen atoms.

Indeed, the naphthalene diimide unit may be bonded to two N,N′-disubstituted bis(pyridinium) units as is the case when it satisfies the formula (II-b) or to a single N,N′-disubstituted bis(pyridinium) unit as is the case when it satisfies the formula (II-a).

In the present invention, the alkyl group (of the R, R¹, R² and R′ groups) denotes a linear or branched group, comprising from 1 to 20 carbon atoms, and preferably from 1 to 3 carbon atoms, said group being optionally fluorinated or perfluorinated, and possibly being interrupted by one or more heteroatoms, for example by one or more oxygen, sulfur or nitrogen atoms.

Methyl groups and electron-attracting alkyl groups such as trifluoromethyl are preferred.

In the present invention, the aryl group (of the R and R′ groups) denotes an aromatic group comprising from 1 to 20 carbon atoms, and preferably from 1 to 6 carbon atoms.

In the present invention, the alkenyl group (of the R and R′ groups) denotes a group comprising at least one alkene function, said group comprising from 1 to 20 carbon atoms, and preferably from 1 to 5 carbon atoms.

In the present invention, the alkynyl group (of the R and R′ groups) denotes a group comprising at least one alkyne function, said group comprising from 1 to 20 carbon atoms, and preferably from 1 to 5 carbon atoms.

The redox ionic compound may comprise several naphthalene diimide units and/or several N,N′-disubstituted bis(pyridinium) units.

When the redox ionic compound comprises several naphthalene diimide units and several N,N′-disubstituted bis(pyridinium) units, they are preferably alternating [e.g. (II-b)/(I-b)/(II-b)/(I-b)/(II-b)/(I-b)/etc.].

In a preferred embodiment of the invention, the redox ionic compound has a theoretical bulk capacity of at least 80 mAh/g approximately, and more preferably of at least 100 mAh/g approximately.

The theoretical bulk capacity of the redox ionic compound depends on its molar mass and on the number of electrons that it can exchange. Thus, it is possible to determine its theoretical bulk capacity as a function of these two parameters.

The redox ionic compound, due to the ionic nature thereof, is in the form of a salt.

In particular, it comprises one or more anions A^{a-} chosen from inorganic anions and organic anions, a representing the valence of the anion, with 1≤a≤3.

As examples of inorganic anions, mention may be made of fluoride (F⁻), chloride (Cl⁻), bromide (Br⁻), iodide (I⁻), tetrafluoroborate (BF₄⁻), metaborate (BO₂⁻), borate (BO₃⁻), perchlorate (ClO₄⁻), fluorate (FO₃⁻), nitrate (NO₃⁻), bis(oxalato)borate [B(C₂O₄)₂⁻], sulfate (SO₄²⁻), disulfate (S₂O₇²⁻), thiosulfate (S₂O₃)²⁻, dithionate (S₂O₆²⁻), phosphate (PO₄³⁻), pyrophosphate (P₂O₇⁴⁻), polyphosphate ((PO₃)⁻ᵣ, r>3), thiosulfate (S₂O₃)², or one of the mixtures thereof.

As examples of organic anions, mention may be made of bis(trifluoromethanesulfonyl)imide (TFSI), bis(fluorosulfonyl)imide (FSI), thiocyanate (SCN⁻), cyanate (CN⁻), formate (HCO₃⁻), acetate (CH₃COO⁻), oxalate (C₂O₄²⁻), dicyanamide (N(CN)₂⁻), tricyanomethanate (C(CN)₃⁻), mellitate (C₁₂H₆O₁₂⁶⁻), tetraaryl borate (BAr₄⁻, with Ar=phenyl or pentafluorophenyl), or one of the mixtures thereof.

The light anions (i.e. having a molar mass Mw<100 g/mol approximately) such as F⁻, Cl⁻, SCN⁻, OCN⁻, BO₂⁻, BO₃⁻, PO₄³⁻, HCO₃⁻, N(CN)₂⁻ or C(CN)₃⁻ are preferred, and Cl⁻ is particularly preferred.

The redox ionic compound preferably has a number-average molar mass ranging from 50 g/mol to 400 g/mol approximately, per electron exchanged.

According to one particularly preferred embodiment of the invention, the redox ionic compound used in the invention comprises or consists of at least one ionic compound of formula (III-a) or (III-b) below:

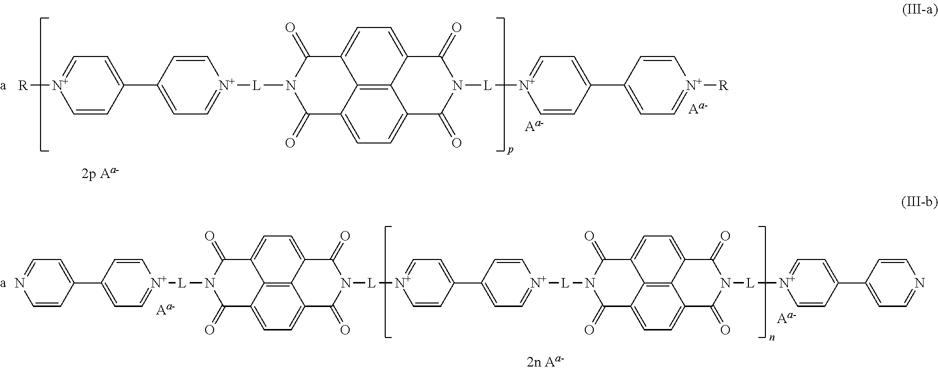

in which R, A, a and L have the same definitions as above, p is such that 1≤p≤10 000 and n is such that 1 n 10 000.

p is preferably such that 1≤p≤10, and more preferably 1≤p≤3; and n is preferably such that 1≤n≤10, and more preferably 1≤n≤3.

In particular, the redox ionic compound comprises or consists of at least one ionic compound of formula (III-a₁) or (III-b₁) below:

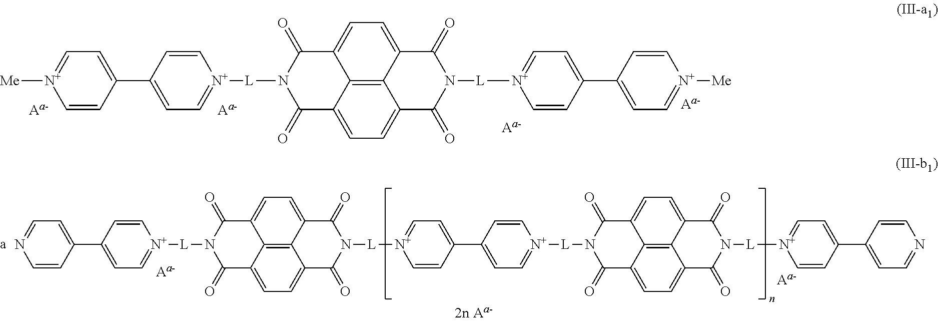

in which L is an alkylene group having 3 carbon atoms (i.e. —CH₂—CH₂—CH₂—), 1 n 3, and A and a have the same definitions as above, and preferably A^{a-} is chosen from Cl⁻, TFSI⁻, Br⁻ and one of the mixtures thereof.

When p>1 [respectively when n>1], the redox ionic compound is an oligomer or a polymer. It may in particular comprise several ionic compounds of formula (III-a) [respectively of formula (III-b)] each of the ionic compounds of formula (III-a) [respectively of formula (III-b)] having a different chain length (i.e. different values of p) [(respectively different values of n)]. It should be noted that each of the ionic compounds of formula (III-a) [respectively of formula (III-b)] having a different chain length is electrochemically active.

According to one preferred embodiment, the dispersion in sizes of the polymers or oligomers present in the redox ionic compound varies from 1 to 10 approximately.

The redox ionic compound may be prepared by bringing 4,4′-bipyridine into contact with 1,4,5,8-naphthalenetetracarboxylic anhydride.

According to one preferred embodiment, the redox ionic compound is prepared according to the following synthesis route A):
reacting 4,4′-bipyridine with an X-L-NH₂ compound, L being as defined previously, and X being a leaving group enabling the nucleophilic substitution of a nitrogen atom of the 4,4′-bipyridine by the -L-NH₂ group,
reacting the compound obtained previously with 1,4,5,8-naphthalenetetracarboxylic anhydride, and
optionally reacting the compound obtained previously with an R—Y compound, R being as defined previously, and Y being a leaving group enabling the nucleophilic substitution of at least one remaining nitrogen atom of the 4,4′-bipyridine by the —R group, or

according to the following synthesis route B):
reacting 1,4,5,8-naphthalenetetracarboxylic anhydride with a Z-L-NH₂ compound, for the amidification of the latter, L being as defined previously, and Z being a leaving group or precursor of a leaving group subsequently enabling the nucleophilic substitution of at least one nitrogen atom of the 4,4′-bipyridine by the -L-(1,4,5,8-naphthalenetetracarboxylic diimide) radical,
reacting the compound obtained previously with 4,4′-bipyridine so as to carry out the nucleophilic substitution of at least one nitrogen atom of the 4,4′-bipyridine by the -L-(1,4,5,8-naphthalenetetracarboxylic diimide) group, and
optionally reacting the compound obtained previously with an R—Y compound, R being as defined previously, and Y being a leaving group enabling the nucleophilic substitution of at least one remaining nitrogen atom of the 4,4′-bipyridine by the —R group.

The route A) may for example result in an ionic compound of formula (III-a) or (III-a₁) as defined previously.

The route B) may for example result in an ionic compound of formula (III-b) or (III-b₁) as defined previously.

The synthesis routes A) and B) may be illustrated in scheme 1 below:

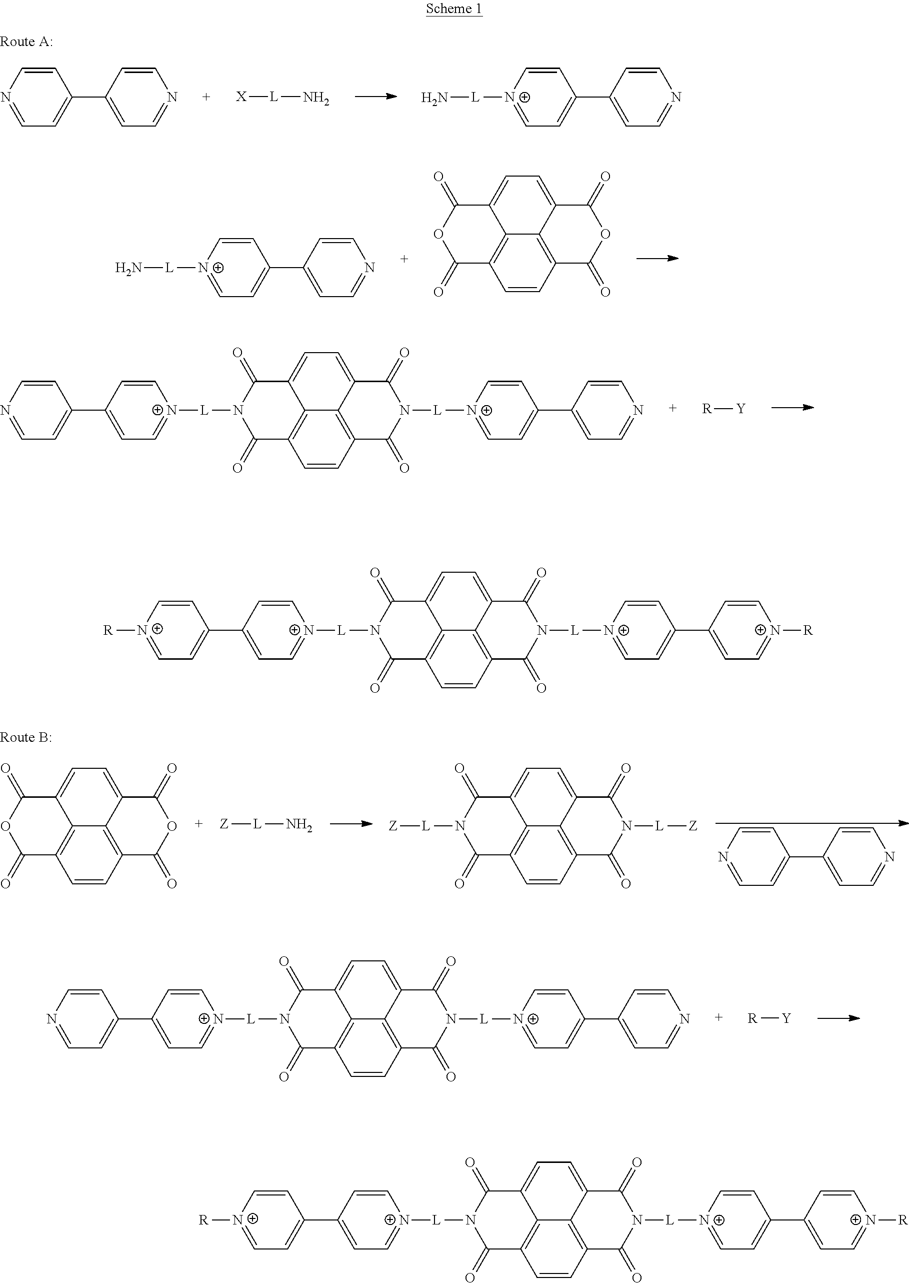

Another synthesis route that may be used to result in a redox ionic compound as defined in the invention and that is similar to route A) is described in Kim et al., J. Mater. Chem., 2012, 22, 13558-13563.

X is preferably a halogen atom, such as for example a bromine (Br) or chlorine (Cl) atom.

Y is preferably a halogen atom, such as for example an iodine (I) atom.

Z is preferably a halogen atom, such as for example a bromine (Br) or chlorine (Cl) atom or an alcohol.

When Z is an alcohol, it may then be esterified by a tosyl, mesityl or trifluoromethanesulfonyl group.

A second subject of the invention is a negative electrode comprising a composite material including a negative electrode active material, optionally a binder, and optionally an agent that imparts electron conductivity, characterized in that the negative electrode active material is a redox ionic compound in accordance with the first subject of the invention.

Preferably, the composite material includes, relative to the total mass of the composite material:
at least 50% by weight approximately of a redox ionic compound in accordance with the first subject of the invention,
from 0% to 50% by weight approximately of a binder,
from 0% to 30% by weight approximately of an agent that imparts electron conductivity,
from 0% to 30% by weight approximately of a salt that imparts ion conductivity, and
from 0% to 30% by weight approximately of a solvent within which the salt that imparts ion conductivity used in the aqueous electrolyte and/or in the negative electrode is soluble.

In one particularly advantageous embodiment, the composite material includes, relative to the total mass of the composite material:

(i) from 55% to 90% by weight approximately of a redox ionic compound in accordance with the first subject of the invention,

(ii) from 0.1% to 10% by weight approximately of a binder,

(iii) from 1% to 25% by weight approximately of an agent that imparts electron conductivity,

(iv) from 0% to 30% by weight approximately of a salt that imparts ion conductivity, and

(v) from 0% to 30% by weight approximately of a solvent within which the salt that imparts ion conductivity used in the negative electrode is soluble.

The agent that imparts electron conductivity that is suitable for the present invention is preferably chosen from carbon black, SP carbon, acetylene black, carbon fibres and nanofibres (e.g. vapour-grown carbon fibres VGCF-S), carbon nanotubes, reduced graphene oxide, graphene oxide, graphite, metal particles and fibres and one of the mixtures thereof.

Among said aforementioned agents that impart electron conductivity, carbon black (e.g. Ketjen black carbon black) or SP carbon is particularly preferred.

The binder may be chosen from copolymers and homopolymers of ethylene; copolymers and homopolymers of propylene; homopolymers and copolymers of ethylene oxide (e.g. PEO, copolymer of PEO), of methylene oxide, of propylene oxide, of epichlorohydrin or of allyl glycidyl ether, and mixtures thereof; halogenated polymers such as homopolymers and copolymers of vinyl chloride, of vinylidene fluoride (PVDF), of vinylidene chloride, of tetrafluoroethylene or of chlorotrifluoroethylene, copolymers of vinylidene fluoride and of hexafluoropropylene (PVDF-co-HFP) or mixtures thereof; polyacrylates such as polymethyl methacrylate; polyalcohols, such as polyvinyl alcohol (PVA); electron-conducting polymers, such as polyaniline, polypyrrole, polyfluorenes, polypyrenes, polyazulenes, polynaphthalenes, polyacetylenes, poly(p-phenylene-vinylene), polycarbazoles, polyindoles, polyazepines, polythiophenes, poly(p-phenylene sulfide) or mixtures thereof; polymers of cationic type, such as polyethyleneimine (PEI), polyaniline in the emeraldine salt (ES) form, poly(quaternized N-vinylimidazole), poly(acrylamide-co-diallyldimethylammonium chloride) (AMAC) or mixtures thereof; biobased binders such as gelatin, agar-agar, carrageenans, pectin or mixtures thereof; and one of the mixtures thereof.

The binder is preferably a homopolymer of ethylene tetrafluoride (PTFE) or a biobased binder.

The salt that imparts ion conductivity may be chosen from a salt of an alkali metal, of an alkaline-earth metal, of aluminium and of the ammonium ion.

The salt of an alkali metal or of an alkaline-earth metal may be chosen from a sodium, lithium, potassium, magnesium, calcium or barium salt.

As examples of salts of an alkali metal or of an alkaline-earth metal, mention may be made of sodium, lithium, potassium, magnesium, calcium or barium perchlorates, sodium, lithium, potassium, magnesium, calcium or barium nitrates, sodium, lithium, potassium, magnesium, calcium or barium chlorides, sodium, lithium, potassium, magnesium, calcium or barium bromides, sodium, lithium, potassium, magnesium, calcium or barium sulfates and sodium, lithium, potassium, magnesium, calcium or barium phosphates.

The solvent within which the salt that imparts ion conductivity used in the negative electrode is soluble may be an aqueous liquid which preferably comprises at least 70% by volume approximately of water, and more preferably 90% to 100% by volume approximately of water, relative to the total volume of liquid in said solvent.

When the proportion of water in the solvent is less than 100% by volume approximately, the solvent may moreover comprise an organic solvent, notably chosen from dimethyl sulfoxide and ethanol (in particular biobased ethanol).

The solvent preferably has a pH varying between 3 and 10, and preferably a neutral pH.

Seawater is very particularly preferred as solvent within which the salt that imparts ion conductivity used in the negative electrode is soluble.

The negative electrode may further comprise a current collector which is coated on its surface with (or bears) a composite material as defined in the invention.

The composite material may be in the form of a film or in the form of a compact powder, borne by the current collector, and preferably in the form of a film.

The current collector is preferably composed of a conductive material, more particularly of a carbon-based material (in the form of fabric, felt, or mat formed by the entanglement of graphite fibres or sheets) or of a metal material which may be chosen from aluminium, nickel, stainless steel and titanium.

The metal material may be in the form of a metal foil, a metal mesh or a metal foam optionally covered with a carbon film.

The thickness of the current collector generally varies from 5 to 50 μm approximately.

Preferably, the composite material of the invention has a thickness ranging from 20 μm to 5 mm approximately.

In one particularly advantageous embodiment of the invention, the negative electrode comprises a surface amount of redox ionic compound ranging from 5 mg/cm² to 200 mg/cm² approximately.

The negative electrode may be prepared:

a) by mixing at least one redox ionic compound with at least one agent that imparts electron conductivity and at least one binder, to obtain a paste of composite material, notably in the form of a film, and

b) by applying said electrode paste to at least one support.

The binder and the agent that imparts electron conductivity are as defined in the present invention.

Step a) may be carried out by extrusion or by milling, notably with the aid of a mortar. It is in particular carried out by manual milling or by milling with the aid of a ball mill (milling well known under the term “ball-milling”). Milling with the aid of a ball mill is preferred.

Step b) may be carried out by laminating, pressing or coating.

The support may be a current collector as defined previously and/or a backing film.

As example of backing film, mention may be made of a plastic film of silicone-coated polyethylene terephthalate (PET) type.

A third subject of the invention is a battery comprising:
a negative electrode,
a positive electrode,
a porous separator inserted between said positive and negative electrodes, and
an aqueous liquid electrolyte impregnating said separator,

characterized in that the negative electrode is in accordance with the second subject of the invention.

The positive electrode may comprise or consist of:

1) a composite material including a positive electrode active material chosen from redox organic compounds and hybrid compounds of metal-organic type, optionally an agent that imparts electron conductivity and optionally a binder, said composite material possibly being supported by a current collector, or
2) an oxide, phosphate or sulfate of transition metals or one of the combinations thereof.

In the first embodiment 1), an organic battery with an aqueous electrolyte is obtained.

As examples of redox organic compounds and of hybrid compounds of metal-organic type, mention may be made of:
complexes of Fe, Mn and Cu with redox-active or non-redox-active ligands, derivatives thereof such as the family of Prussian blues, metallocenes or phthalocyanines, or else complexes of Fe, Mn and Cu in which one of the ligands is 2,2′-bipyridine,
quinone-based compounds and the polymer derivatives thereof,
compounds containing the 2,2,6,6-tetramethylpiperidin-1-yl)oxy (TEMPO) group and the polymer derivatives thereof, or
pigments such as for example Fast Blue, indigo or thioindigo.

In the second embodiment 2), an inorganic-organic hybrid battery with an aqueous electrolyte is obtained.

As an example of an oxide, phosphate, or sulfate of transition metals or one of the combinations thereof, mention may be made of Na_{y}LiₓMn₂O₄ (0<x<1, 0<y<1, and x+y≤1.1), the NaMPO₄ system, the NaM₂(PO₄)₃ system, the Na₂MPO₄F system preferably with M=Fe, Mn for reasons of cost and abundance (but M may also be inter alia chosen from Co, Ni, Cr, V, Ti, Cu, Zr, Nb, W and Mo), the material Li(Na)Co_{1/3}Ni_{1/3}Mn_{1/3}O₂, NaMnO₂ (birnessite structure) optionally doped with one or more metals such as Li or Al, NaMn₉O₁₈ or Na₂Mn₃O₇.

The aqueous liquid electrolyte may comprise a salt of an alkali metal, of an alkaline-earth metal, of aluminium or of the ammonium ion in water or may very simply consist of seawater.

The salt of an alkali metal or of an alkaline-earth metal may be chosen from a sodium, lithium, potassium, magnesium, calcium and barium salt.

As examples of salts of an alkali metal or of an alkaline-earth metal, mention may be made of sodium, lithium, potassium, magnesium, calcium or barium perchlorates, sodium, lithium, potassium, magnesium, calcium or barium nitrates, sodium, lithium, potassium, magnesium, calcium or barium chlorides, sodium, lithium, potassium, magnesium, calcium or barium bromides, sodium, lithium, potassium, magnesium, calcium or barium sulfates and sodium, lithium, potassium, magnesium, calcium or barium phosphates.

Seawater is very particularly preferred.

The aqueous liquid electrolyte preferably has a pH varying between 3 and 10, and preferably a neutral pH.

The aqueous liquid electrolyte preferably comprises at least 70% by volume approximately of water and more preferably 90% to 100% by volume approximately of water, relative to the total volume of liquid in the aqueous liquid electrolyte.

When the proportion of water in the liquid of the aqueous liquid electrolyte is less than 100% by volume approximately, the aqueous liquid electrolyte may moreover comprise an organic co-solvent, notably chosen from dimethyl sulfoxide, an alcohol (in particular biobased ethanol) and one of the mixtures thereof.

The aqueous liquid electrolyte completely saturates the porous separator in order to impregnate the porosity thereof.

The choice of the porous separator is not limiting and this is well known to those skilled in the art.

The porous separator may be made of a porous material that is not electron conducting, generally made of a polymer material based on polyolefin (e.g. polyethylene) or made of fibres (e.g. glass fibres or wood fibres, cellulose fibres).

The battery in accordance with the third subject of the invention may be prepared according to the following steps:
preparing an aqueous liquid electrolyte as defined in the present invention, notably by mixing water with a salt of an alkali metal, of an alkaline-earth metal, of aluminium or of the ammonium ion,
assembling a positive electrode, a negative electrode and a porous separator, as defined in the present invention, and
impregnating the assembly as obtained in the preceding step with the aqueous liquid electrolyte.

A fourth subject of the invention is a redox ionic compound characterized in that it comprises or consists of at least one ionic compound of formula (III-a), (III-a₁), (III-b) or (III-b₁) as defined in the first subject of the invention, with the exception of the compounds of formula (III-a) for which L is a linear alkylene chain having 2 or 10 carbon atoms.

Preferably, the redox ionic compound comprises or consists of at least one ionic compound of formula (III-a), (III-a₁), (III-b), (III-b₁) as defined in the first subject of the invention, with L being a preferably linear, alkylene chain having from 3 to 6 carbon atoms, and more preferentially still 3, 4 or 5 carbon atoms.

The present invention is illustrated by the examples below, to which, however, it is not limited.

### EXAMPLES

The raw materials used in the examples are listed below:
carbon black, Carbon super P, TIMCAL,
polytetrafluoroethylene (PTFE), Aldrich,
1,4,5,8-naphthalenetetracarboxylic anhydride, Aldrich,
triethylamine, Aldrich,
3-bromopropylamine, Aldrich,
acetic acid, Carlo Erba,
anhydrous dimethylformamide (DMF), Aldrich,
lithium bis(trifluoromethanesulfonyl)imide (LiTFSI), Aldrich,
4,4′-bipyridine, Aldrich,
acetonitrile, Carlo Erba,
o-dichlorobenzene, Aldrich,
dimethylaminopyridine (DMAP), Aldrich,
imidazole, Aldrich,
NaClO₄, Aldrich,
MeI, Aldrich,
Mg(ClO₄)₂, Aldrich
4-hydroxy-TEMPO-benzoate, Sigma Aldrich.

Unless otherwise indicated, all the materials were used as received from the manufacturers.

### Example 1

### Preparation of the Redox Ionic Compounds 1 and 2

1.1 Preparation of the Redox Ionic Compound 1

The redox ionic compound 1 satisfies the following formula:

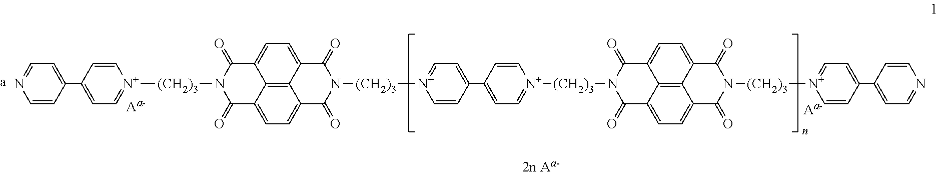

in which A^{a-} is a mixture of TFSI⁻ and Br⁻, and n is such that 1 n 3.

1.072 g of 1,4,5,8-naphthalenetetracarboxylic anhydride were mixed under a nitrogen atmosphere with 3.280 g of 3-bromopropylamine in the presence of 2 ml of triethylamine and 20 ml of acetic acid. The resulting mixture was brought to reflux for 24 h. The intermediate product formed was isolated in the following manner: the precipitate was filtered and washed thoroughly with water and methanol (MeOH) to result in the intermediate product with 77% yield.

In a sealed tube, 0.5 g of intermediate product formed above was mixed with 0.154 g of 4,4′-bipyridine in 10 ml of anhydrous dimethylformamide (DMF), then the resulting mixture was heated at 130° C. for 2 days. After 2 days, 2 g of lithium bis(trifluoromethanesulfonyl)imide (LiTFSI) in 5 ml of DMF were added to the stirred solution. The mixture was left for another 2 days at 130° C. After cooling to ambient temperature, the brown precipitate was filtered and washed thoroughly with dichloromethane (DCM) to give 0.18 g of the final compound.

The final addition of LiTFSI may be omitted by leaving the mixture for 4 days at 130° C., by filtering and by washing the precipitate with dichloromethane (DCM). This procedure results in the same amount of redox-active fraction of the compound 1, but with only bromide ions as counter-anions.

1.2 Preparation of the Redox Ionic Compound 2

The redox ionic compound 2 satisfies the following formula:

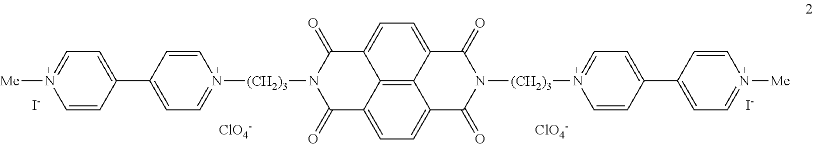

2 g of 4,4′-bipyridine were mixed with 0.280 g of 3-bromopropylamine in the presence of 20 ml of acetonitrile. The resulting mixture was brought to reflux for 1.5 h. The intermediate product formed was isolated in the following manner: the mixture was cooled to ambient temperature, the resulting white precipitates were collected by filtration and dried under vacuum to give an intermediate product in the form of a white solid (yield of 47%).

0.213 g of intermediate product formed above was mixed with 0.113 g of 1,4,5,8-naphthalenetetracarboxylic anhydride in 5 ml of o-dichlorobenzene in the presence of 0.064 g of DMAP and 0.077 g of imidazole, then the resulting mixture was heated at 80° C. for 12 h. After cooling, the resulting precipitates were collected by filtration. The red solid collected was dissolved in water (50 ml), then the solution was washed with dichloromethane (DCM) to remove the water-insoluble residues. An excess of powdered sodium perchlorate was added to the separated water layer. A second intermediate product in the form of a white precipitate was collected by filtration and dried under vacuum (yield of 63%).

A mixture of the second intermediate product formed above (173 mg) and iodomethane (50 μl) in acetonitrile (6 ml) was heated at reflux at 90° C. for 24 h. After cooling to ambient temperature, the resulting precipitates were collected by filtration and washed with acetonitrile. The resulting red precipitates were dried under vacuum (yield of 52%).

1.3 Preparation of the Redox Ionic Compound 3

The redox ionic compound 3 satisfies the following formula:

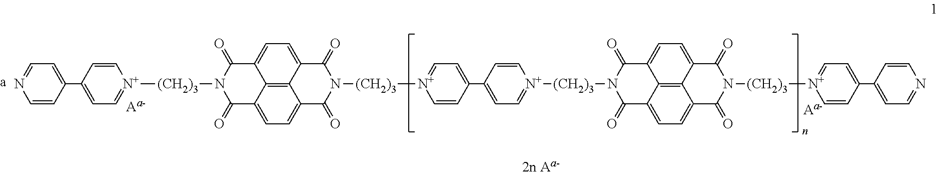

in which A^{a-} is Cl⁻, and n is such that 1 n 3.

50 mg of the redox ionic compound 1 were added and dispersed in a saturated solution of NaCl. The suspension was stirred for 4 days at 50° C. Next, the precipitate obtained was washed several times with water, and dried at 60° C. under vacuum overnight to obtain 40 mg of 3.

### Example 2

### Electrochemical Performance of the Redox Ionic Compound 1

2.1 Preparation of the Negative Electrode

25 mg of Ketjen black carbon black and 70 mg of redox ionic compound 1 as prepared in Example 1.1 were mixed by manual co-milling in a mortar. 5 mg of PTFE were then added to this mixture and the resulting mixture was co-milled, which makes it possible to form a film of composite material.

The film thus obtained was then pressed onto a 316L stainless steel mesh at 5 tonnes/cm². The electrode comprised around 10 mg/cm² of redox ionic compound 1. The negative electrode had a total thickness of 100 μm and comprised an amount of 2 mg approximately of redox ionic compound 1.

Table 1 below presents the composition by weight of the negative electrode E-1 obtained:

TABLE 1

| Negative | Carbon | | Redox ionic |
|---|---|---|---|
| electrode | black (%) | PTFE (%) | compound 1 (%) |
| E-1 | 25 | 5 | 70 |

2.2 Electrochemical tests on cells C¹ and C²

The electrochemical tests were carried out under a nitrogen atmosphere in glass cells comprising three electrodes and 10 ml of electrolyte.

The three electrodes of the cell C¹ were the following:
a working electrode consisting of the negative electrode E-1 as prepared in Example 2.1,
a reference electrode consisting of a calomel electrode (SCE), and
a counter electrode consisting of a mixture of 95% by weight of carbon (Ketjen Black) and 5% by weight of PTFE and having a capacity systematically oversized by a factor of two to four, relative to the working electrode.

The aqueous liquid electrolyte of the cell C¹ was a 1.25M aqueous solution of Mg(ClO₄)₂.

In the cell, the electrodes are immersed in a large excess of electrolyte and are approximately 1 cm from one another. It is therefore not necessary to use a separator.

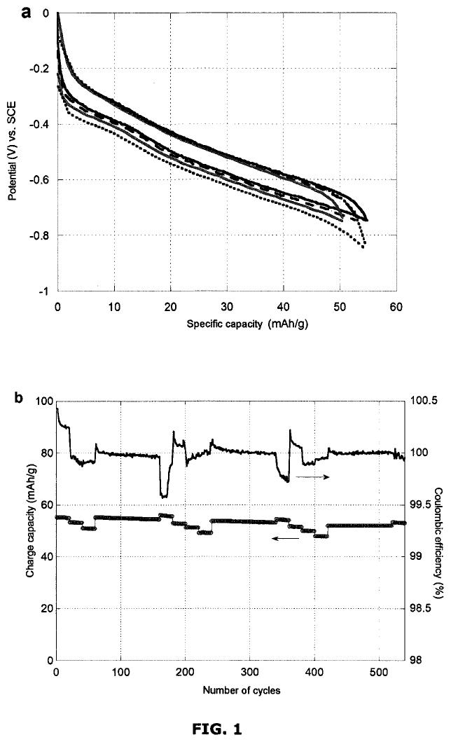

FIG. 1a shows the potential versus SCE (in volts, V) as a function of the specific capacity (in mAh/g) at various currents 0.3 A/g (curve with the black solid line), 0.6 A/g (curve with the large dots), 1.2 A/g (curve with the grey solid line), and 2.4 A/g (curve with the small dots) for the cell C¹.

FIG. 1b shows the charge capacity (in mAh/g) (bottom curve) and coulombic efficiency (in %) (top curve) as a function of the number of cycles for the cell C¹, when the following cycling protocol denoted by P¹ was carried out: galvanostatic cycling between 0 and −0.75 V with successive sequences of cycles for a current of 0.3 A/g, 0.6 A/g and 1.2 A/g, followed by galvanostatic cycling between 0 and −0.90 V with a sequence of 100 cycles at a current of 2.4 A/g, followed by galvanostatic cycling between 0 and −0.75 V with a sequence of 20 cycles for a current of 0.3 A/g, followed by a period at a constant potential of −0.75 V for one minute.

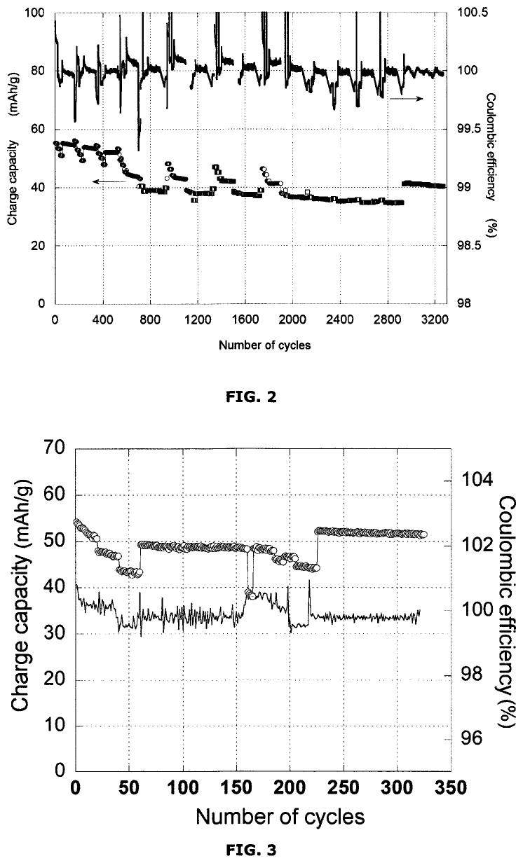

FIG. 2 shows the charge capacity (in mAh/g) (bottom curve) and coulombic efficiency (in %) (top curve) as a function of the number of cycles for the cell C¹, when the cycling protocol P¹ as defined above was carried out until the 520^{th} cycle; followed by a subsequent cycling protocol P² carried out until the 1853^{rd} cycle: galvanostatic cycling between 0 and −0.90 V with successive sequences of 20 cycles for a current of 0.3 A/g, 0.6 A/g and 1.2 A/g, followed by galvanostatic cycling between 0 and −0.90 V with a sequence of 100 cycles at a current of 2.4 A/g, followed by galvanostatic cycling between 0 and −0.75 V with a sequence of 20 cycles for a current of 0.3 A/g, followed by a period at a constant potential of −0.75 V for one minute; followed by a subsequent cycling protocol P³ carried out until the end: galvanostatic cycling between 0 and −0.85 V with a sequence of 100 cycles at a current of 2.4 A/g.

A cell C² in which the three electrodes were identical to those used for the cell C¹ and the aqueous liquid electrolyte was unfiltered water from the Atlantic Ocean (instead of the 1.25M aqueous solution of Mg(ClO₄)₂) was tested.

FIG. 3 shows the charge capacity (in mAh/g) (top curve) and coulombic efficiency (in %) (bottom curve) as a function of the number of cycles for the cell C², when the cycling protocol P¹ as defined above was carried out, except that between the 161^{st} to 165^{th} cycles, the current was 0.3 A/g with galvanostatic cycling between 0 and −0.65 V, followed by a period at a constant potential of −0.65 V for one minute.

### Example 3

### Electrochemical Performance of the Redox Ionic Compound 2

3.1 Preparation of the Negative Electrode

25 mg of Ketjen black carbon black and 70 mg of redox ionic compound 2 as prepared in Example 1.1 were mixed by manual co-milling in a mortar. 5 mg of PTFE were then added to this mixture and the resulting mixture was co-milled, which makes it possible to form a film of composite material.

The film thus obtained was then pressed onto a 316L stainless steel mesh at 5 tonnes/cm². The electrode comprised around 10 mg/cm² of redox ionic compound 2. The negative electrode had a total thickness of 100 μm and comprised an amount of 2 mg approximately of redox ionic compound 2.

Table 2 below presents the composition by weight of the negative electrode E-2 obtained:

TABLE 2

| Negative | Carbon | | Redox ionic |
|---|---|---|---|
| electrode | black (%) | PTFE (%) | compound 2 (%) |
| E-2 | 25 | 5 | 70 |

3.2 Electrochemical Tests on Cells C³, C^{A}, C^{B} and C^{C}

The electrochemical tests were carried out under a nitrogen atmosphere in glass cells comprising three electrodes and 10 ml of electrolyte.

The three electrodes of the cell C³ were the following:
a working electrode consisting of the negative electrode E-2 as prepared in Example 3.1,
a reference electrode consisting of a calomel electrode (SCE), and
a counter electrode consisting of a mixture of 95% by weight of carbon (Ketjen Black) and 5% by weight of PTFE and having a capacity systematically oversized by a factor of two to four, relative to the working electrode.

The aqueous liquid electrolyte of the cell C³ was a 2.5M aqueous solution of NaClO₄.

In the cell, the electrodes are immersed in a large excess of electrolyte and are approximately 1 cm from one another. It is therefore not necessary to use a separator.

The cell C³ was compared to cells C^{A}, C^{B} and C^{C} that are not part of the invention, and the features of which are the following, relative to the cell C³:
the cell C^{A} comprised a mixture A that is not part of the invention consisting of a 4,4′-bipyridine that is N,N′-disubstituted by a methyl and a naphthalene diimide that is disubstituted by a methyl, having a 2:1 molar ratio, the mixture A having the following formula:

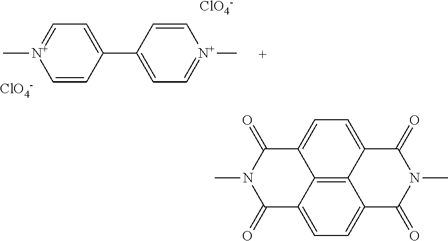

instead of the redox ionic compound 2,
the cell C^{B} comprised a 4,4′-bipyridine N,N′-disubstituted by a methyl that is not part of the invention instead of the redox ionic compound 2, and
the cell C^{C} comprised a naphthalene diimide disubstituted by a methyl that is not part of the invention instead of the redox ionic compound 2.

The 4,4′-bipyridine N,N′-disubstituted by a methyl was prepared from the corresponding commercial chlorinated compound (Sigma Aldrich), by dissolving said compound in water and by re-precipitating it in the presence of sodium perchlorate.

The naphthalene diimide disubstituted by a methyl was prepared according to the process as described in Sci. China Chem., 2012, 55, 10.

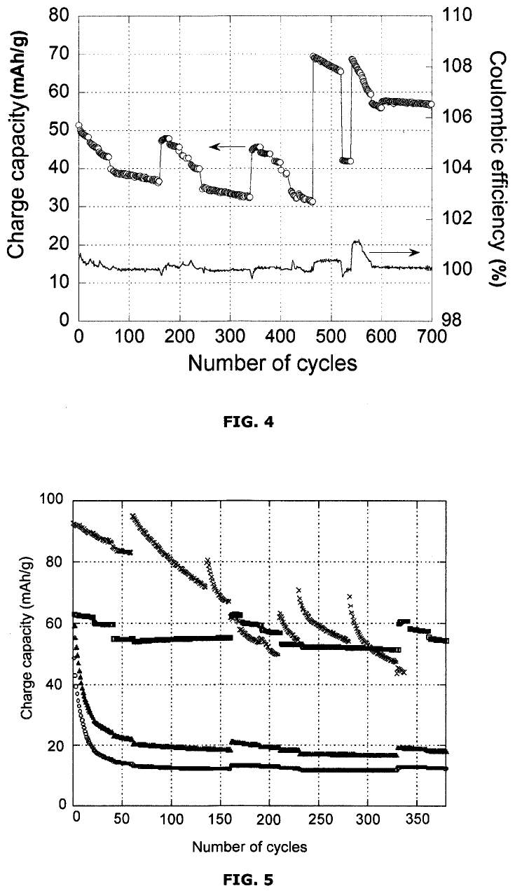

FIG. 4 shows the charge capacity (in mAh/g) (top curve) and coulombic efficiency (in %) (bottom curve) as a function of the number of cycles for the cell C³, when the cycling protocol P¹ as defined in Example 2.2 was carried out until the 470^{th} cycle, followed by galvanostatic cycling between 0 and −1.20 V until the 520^{th} cycle for a current of 2.4 A/g, followed by galvanostatic cycling between 0 and −0.75 V until the 550^{th} cycle for a current of 0.3 A/g, followed by galvanostatic cycling between 0 and −1.10 V until the 580^{th} cycle for a current of 1.2 A/g, followed by galvanostatic cycling between 0 and −1.20 V until the end for a current of 2.4 A/g.

FIG. 5 shows the charge capacity (in mAh/g) as a function of the number of cycles of the cells C³ (curve with squares), C^{A} (curve with triangles), C^{B} (curve with circles) and C^{C} (curve with crosses), when the following cycling protocol was carried out: galvanostatic cycling between 0 and −0.75 V with successive sequences of 20 cycles for a current of 0.3 A/g, 0.6 A/g and 1.2 A/g, followed by galvanostatic cycling between 0 and −0.90 V with a sequence of 100 cycles at a current of 2.4 A/g, followed by a loop.

The respective molar proportions of naphthalene diimide and of N,N′-disubstituted 4,4′-bipyridine in the mixtures A and B were the same as in the redox ionic compound 2.

As can clearly be seen in FIG. 5, the electrochemical performance is significantly worse when a mixture of two separate compounds, a naphthalene diimide and an N,N′-disubstituted 4,4′-bipyridine, is used, compared to a redox ionic compound as defined in the invention which comprises both the two aforementioned compounds optionally in the form of repeat units.

### Example 4

### Battery in Accordance with the Invention

4.1 Preparation of the Negative Electrode

25 mg of Ketjen black carbon black and 70 mg of redox ionic compound 1 as prepared in Example 1.1 were mixed by manual co-milling in a mortar. 5 mg of PTFE were then added to this mixture and the resulting mixture was co-milled, which makes it possible to form a film of composite material.

The film thus obtained was then pressed onto a 316L stainless steel mesh at 5 tonnes/cm². The electrode comprised around 10 mg/cm² of redox ionic compound 1. The negative electrode had a total thickness of 100 μm and comprised an amount of 2 mg approximately of redox ionic compound 1.

Table 3 below presents the composition by weight of the negative electrode E-3 obtained:

TABLE 3

| Negative | Carbon | | Redox ionic |
|---|---|---|---|
| electrode | black (%) | PTFE (%) | compound 2 (%) |
| E-3 | 25 | 5 | 70 |

4.2 Preparation of a Battery B-1

A battery B-1 was prepared by assembling three electrodes under a nitrogen atmosphere at ambient temperature:
the negative electrode E-3 obtained in Example 4.1 above,
a positive electrode consisting of 66% by weight of carbon black and 34% by weight of Prussian blue Fe₇(CN)₁₈, and having a capacity oversized by a factor of four, relative to the negative electrode,
a calomel reference electrode (SCE), and
the aqueous liquid electrolyte was unfiltered water from the Atlantic Ocean.

In the battery, the positive and negative electrodes are immersed in a large excess of electrolyte and are approximately 1 cm from one another. It is therefore not necessary to use a separator.

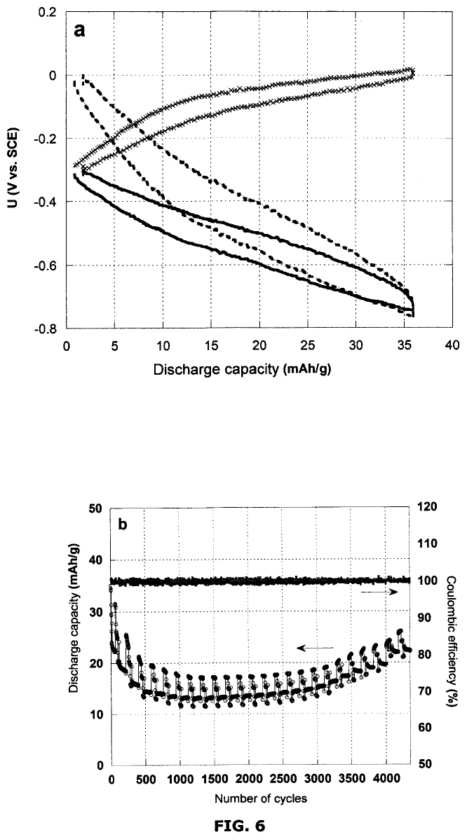

FIG. 6a shows the potential versus SCE (in volts, V) as a function of the discharge capacity (in mAh/g) for the negative electrode (curve with the solid line), for the positive electrode (curve with the crosses), and for the complete battery B-1 (curve with the dotted lines), in the 2^{nd} cycle for a current of 0.225 A/g (nominal rate of 4 C).

FIG. 6b shows the charge capacity (in mAh/g) (bottom curve) and coulombic efficiency (in %) (top curve) as a function of the number of cycles for the complete battery B-1, when the cycling protocol P¹ as defined in Example 2.1 was carried out.

The results of Examples 2-4 show that the redox ionic compounds as used in the invention have good electrochemical performance in terms of capacity, cyclability and efficiency.

### Example 5

### Battery in Accordance with the Invention

5.1 Preparation of the Negative Electrode

25 mg of Ketjen black carbon black and 70 mg of redox ionic compound 1 as prepared in Example 1.1 were mixed by manual co-milling in a mortar. 5 mg of PTFE were then added to this mixture and the resulting mixture was co-milled manually, which makes it possible to form a film of composite material.

The film thus obtained was then cut to the desired size and pressed onto a 316L stainless steel mesh at 5 tonnes/cm². The electrode comprised around 10 mg/cm² of redox ionic compound 1. The negative electrode had a total thickness of 100 μm and comprised an amount of 1 mg approximately of redox ionic compound 1.

Table 4 below presents the composition by weight of the negative electrode E-4 obtained:

TABLE 4

| Negative | Carbon | | Redox ionic |
|---|---|---|---|
| electrode | black (%) | PTFE (%) | compound 1 (%) |
| E-4 | 25 | 5 | 70 |

5.2 Preparation of a Battery B-2

A batterie B-2 was prepared by assembling three electrodes under nitrogen atmosphere at ambient temperature:
the negative electrode E-4 obtained in Example 5.1 above,
a positive electrode consisting of 25% by weight of carbon black, 5% by weight of PTFE and 70% by weight of 4-hydroxy-TEMPO-benzoate, and having a capacity oversized by a factor of 1, relative to the negative electrode,
a calomel reference electrode (SCE), and
the aqueous liquid electrolyte was a saturated solution of NaClO₄.

In the battery, the positive and negative electrodes are immersed in a large excess of electrolyte and are approximately 1 cm from one another. It is therefore not necessary to use a separator.

FIG. 7 shows the charge capacity (in mAh) (top curve) and charge energy (in mW·h) (bottom curve) as a function of the number of cycles for the complete battery B-2 (each of the two electrodes being 1.1 mg approximately), when the voltage of the cell is 1.12 V and the cycling protocol carried out is the following: galvanostatic cycling at a current of 0.075 A/g with a maximum charge voltage of 1.8 V.

### Example 6

### Electrodes in Accordance with the Invention

6.1 Preparation of the Negative Electrode

20 mg or 15 mg of Ketjen black carbon black and 75 mg or 80 mg of redox ionic compound 1 as prepared in Example 1.1 were mixed by co-milling using a ball mill sold under the trade name Pulverisette 7 Classic Line by the company Fritsch. 5 mg of PTFE were then added to this mixture and the resulting mixture was co-milled using the ball mill, which makes it possible to form a film of composite material.

The film thus obtained was then pressed onto a 316L stainless steel mesh at 5 tonnes/cm². The electrode comprised around 10 mg/cm² of redox ionic compound 1. The negative electrode had a total thickness of 100 μm and comprised an amount of 1 mg approximately of redox ionic compound 1.

Table 5 below presents the composition by weight of the negative electrode E-5 or E-6 obtained:

TABLE 5

| Negative | Carbon | | Redox ionic |
|---|---|---|---|
| electrode | black (%) | PTFE (%) | compound (%) |
| E-5 | 20 | 5 | 75 |
| E-6 | 15 | 5 | 80 |

6.2 Electrochemical Tests on Cells C⁵, C⁶ and C¹

The electrochemical tests were carried out under a nitrogen atmosphere in glass cells comprising three electrodes and 10 ml of electrolyte.

The three electrodes of the cell C⁵ (respectively of the cell C⁶) were the following:
a working electrode consisting of the negative electrode E-5 (respectively of the negative electrode E-6) as prepared in Example 6.1,
a reference electrode consisting of a calomel electrode (SCE), and
a counter electrode consisting of a mixture of 90% by weight of carbon (Ketjen Black) and 10% by weight of PTFE and having a capacity systematically oversized by a factor of two to four, relative to the working electrode.

The aqueous liquid electrolyte of the cells C⁵ and C⁶ was a 2.5M solution of NaClO₄.

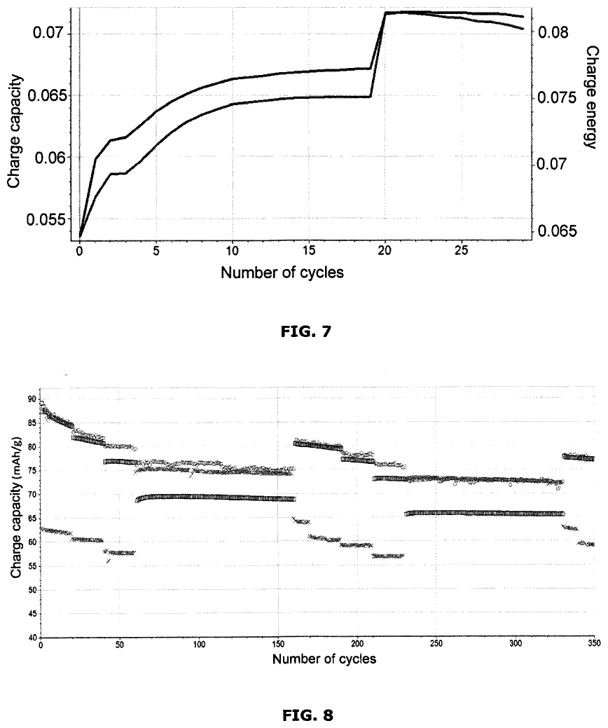

FIG. 8 shows the charge capacity (in mAh/g) as a function of the number of cycles for the cell C¹ as defined previously (curve with the crosses), for the cell C⁵ (curve with the circles) and for the cell C⁶ (curve with the squares) when the following cycling protocol was carried out: galvanostatic cycling with successive looped linkages of two sequences (i) then (ii) then (i): (i) between 0 and −0.75 V with successive sequences of 20 cycles for a current of 0.3 A/g, 0.6 A/g and 1.2 A/g, followed by (ii) between 0 and −0.85 V with a sequence of 100 cycles at a current of 2.4 A/g.

### Example 7

### Electrodes in Accordance with the Invention

7.1 Preparation of the Negative Electrode

25 mg of Ketjen black carbon black and 70 mg of redox ionic compound 3 as prepared in Example 1.3 were mixed by manual co-milling in a mortar. 5 mg of PTFE were then added to this mixture and the resulting mixture was co-milled, which makes it possible to form a film of composite material.

The film thus obtained was then pressed onto a 316L stainless steel mesh at 5 tonnes/cm². The electrode comprised around 10 mg/cm² of redox ionic compound 3. The negative electrode had a total thickness of 100 μm and comprised an amount of 2 mg approximately of redox ionic compound 3.

Table 6 below presents the composition by weight of the negative electrode E-7 obtained:

TABLE 6

| Negative | Carbon | | Redox ionic |
|---|---|---|---|
| electrode | black (%) | PTFE (%) | compound 1 (%) |
| E-7 | 25 | 5 | 70 |

7.2 Electrochemical tests on cells C⁷ and C¹

The electrochemical tests were carried out under a nitrogen atmosphere in glass cells comprising three electrodes and 10 ml of electrolyte.

The three electrodes of the cell C⁷ were the following:
a working electrode consisting of the negative electrode E-7 as prepared in Example 7.1,
a reference electrode consisting of a calomel electrode (SCE), and
a counter electrode consisting of a mixture of 95% by weight of carbon (Ketjen Black) and 5% by weight of PTFE and having a capacity systematically oversized by a factor of two to four, relative to the working electrode.

The aqueous liquid electrolyte of the cell C⁷ was a 2.5M aqueous solution of NaClO₄.

In the cell, the electrodes are immersed in a large excess of electrolyte and are approximately 1 cm from one another. It is therefore not necessary to use a separator.

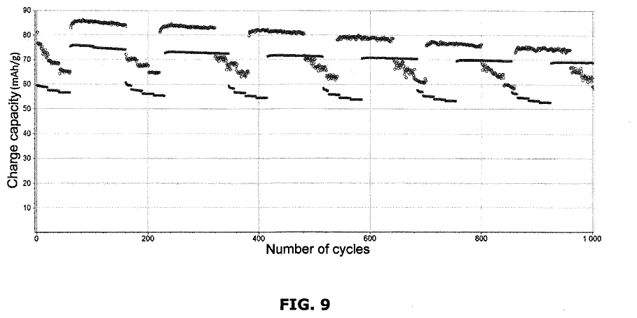

FIG. 9 shows the charge capacity (in mAh/g) as a function of the number of cycles for the cell C¹ as defined previously (curve with the squares), and for the cell C⁷ (curve with the circles) when the following cycling protocol was carried out: galvanostatic cycling with successive looped linkages of two sequences (i) then (ii) such that: (i) between 0 and −0.75 V with successive sequences of 20 cycles for a current of 0.3 A/g, 0.6 A/g and 1.2 A/g, followed by (ii) between 0 and −0.85 V with a sequence of 100 cycles at a current of 2.4 A/g.

1. A redox ionic compound, said redox compound configured to be operable as a negative electrode active material, said redox compound comprising:
at least one naphthalene diimide unit; and
at least one N,N′-disubstituted bis(pyridinium) unit.

2. The redox ionic compound according to claim 1, wherein the N,N′-disubstituted bis(pyridinium) and naphthalene diimide units are coupled within the redox ionic compound by means of a linker denoted by L.

3. The redox ionic compound according to claim 2, wherein the linker L is a saturated or unsaturated carbon chain, an aromatic carbon chain, or a mixture of a saturated or unsaturated carbon chain and an aromatic carbon chain, the aforementioned carbon chains being optionally fluorinated, and possibly containing one or more heteroatoms, for example one or more oxygen or sulfur atoms, said carbon chains having from 2 to 20 carbon atoms.

4. The redox ionic compound according to claim 1, wherein the redox ionic compound comprises one or more anions A′ chosen from inorganic anions and organic anions, a representing the valence of the anion, with 1≤a≤3.

5. The redox ionic compound according to claim 1, wherein the N,N′-disubstituted bis(pyridinium) unit is represented by any one of the chemical formulae (I-a), (I-b) or (I-c) below: 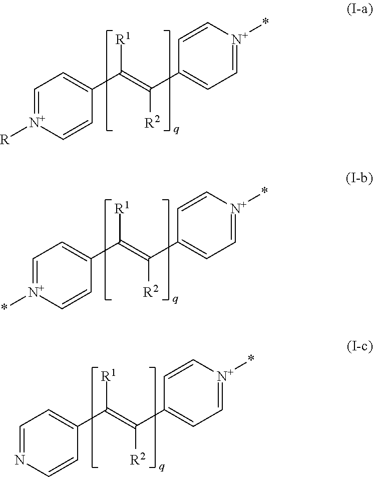
in which the sign * denotes the attachment point of the N,N′-disubstituted bis(pyridinium) unit to a naphthalene diimide unit; R represents an end group chosen from an alkyl group, an alkenyl group, an alkynyl group, and an aryl group, said aforementioned groups being optionally substituted by one or more aromatic groups, optionally fluorinated or perfluorinated, said aforementioned groups possibly containing one or more heteroatoms, for example one or more oxygen, sulfur or nitrogen atoms; R¹ and R², which are identical or different, represent an alkyl group or a cyano group; and q is such that 0≤q≤4.

6. The redox ionic compound according to claim 1, wherein the naphthalene diimide unit is represented by either one of the chemical formulae (II-a) or (II-b) below: 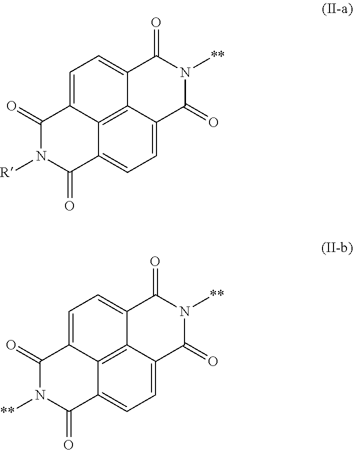
in which the sign ** denotes the attachment point of the naphthalene diimide unit to an N,N′-disubstituted bis(pyridinium) unit and R′ represents an end group chosen from a hydrogen atom, an alkyl group, an alkenyl group, an alkynyl group, and an aryl group, said aforementioned groups being optionally substituted by one or more aromatic groups, optionally fluorinated or perfluorinated, said aforementioned groups possibly containing one or more heteroatoms, for example one or more oxygen, sulfur or nitrogen atoms.

7. The redox ionic compound according to claim 1, wherein the redox ionic compound comprises at least one ionic compound of formula (III-a) or (III-b) below: 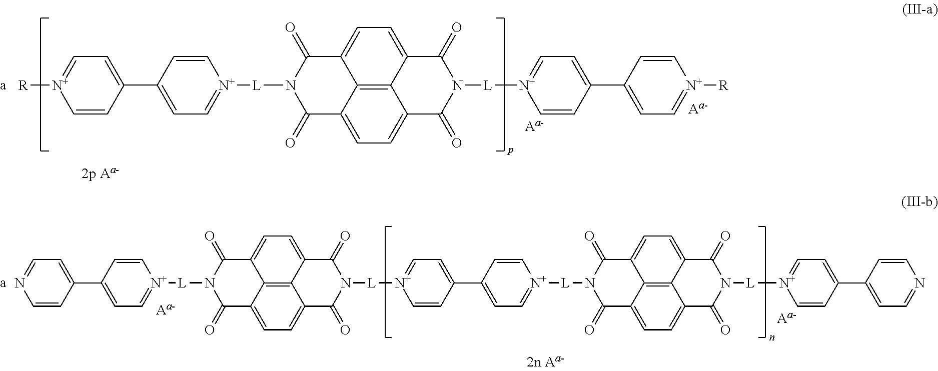
in which R, A, a and L have the same definitions as in claims 2 to 5, p is such that 1≤p≤10 000 and n is such that 1≤n≤10 000.

8. The redox ionic compound according to claim 1, wherein the redox ionic compound comprises at least one ionic compound of formula (III-a₁) or (III-b₁) below: 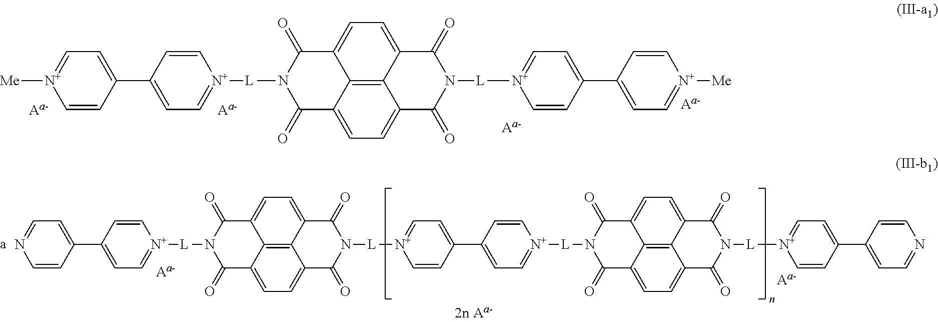
in which L is an alkylene group having 3 carbon atoms, 1≤n≤3 and A^{a-} is chosen from Cl⁻, TFSI⁻, Br⁻ and one of the mixtures thereof.

9. The redox ionic compound according to claim 1, wherein the redox ionic compound has a theoretical bulk capacity of at least 80 mAh/g.

10. A negative electrode comprising:
a composite material including a negative electrode active material,
optionally a binder, and
optionally an agent that imparts electron conductivity, wherein the negative electrode active material is a redox ionic compound as defined in claim 1.

11. The negative electrode according to claim 10, wherein the composite material includes, relative to the total mass of the composite material:
(i) from 55% to 90% by weight of a redox ionic compound as defined in claim 1,
(ii) from 0.1% to 10% by weight of a binder,
(iii) from 1% to 25% by weight of an agent that imparts electron conductivity,
(iv) from 0% to 30% by weight of a salt that imparts ion conductivity, and
(v) from 0% to 30% by weight of a solvent within which the salt that imparts ion conductivity used in the negative electrode is soluble.

12. The negative electrode according to claim 10, wherein the agent that imparts electron conductivity is chosen from carbon black, SP carbon, acetylene black, carbon fibres and nanofibres, carbon nanotubes, reduced graphene oxide, graphene oxide, graphite, metal particles and fibres and one of the mixtures thereof.

13. The negative electrode according to claim 10, wherein the binder is chosen from copolymers and homopolymers of ethylene; copolymers and homopolymers of propylene; homopolymers and copolymers of ethylene oxide, of methylene oxide, of propylene oxide, of epichlorohydrin or of allyl glycidyl ether, and mixtures thereof halogenated polymers; polyacrylates; polyalcohols; electron-conducting polymers; polymers of cationic type; biobased binders; and one of the mixtures thereof.

14. The negative electrode according to claim 10, wherein the negative electrode further comprises a current collector which is coated on its surface with said composite material.

15. A battery comprising:
a negative electrode,
a positive electrode,
a porous separator inserted between said positive and negative electrodes, and
an aqueous liquid electrolyte impregnating said separator,
wherein the negative electrode is as defined in claim 10.

16. The battery according to claim 15, wherein the positive electrode comprises:
1) a composite material including a positive electrode active material chosen from redox organic compounds and hybrid compounds of metal-organic type, optionally an agent that imparts electron conductivity and optionally a binder, said composite material possibly being supported by a current collector, or
2) an oxide, phosphate or sulfate of transition metals or one of the combinations thereof.

17. The battery according to claim 15, wherein the aqueous liquid electrolyte comprises a salt of an alkali metal, of an alkaline-earth metal, of aluminium or of the ammonium ion in water or consists of seawater.

18. A redox ionic compound wherein said redox ionic compound comprises at least one ionic compound of formula (III-a), (III-a₁), (III-b) or (III-b₁) as defined in claim 7, with the exception of the compounds of formula (III-a) for which L is a linear alkylene chain having 2 or 10 carbon atoms.
